SEQUENCE LISTING (10 sequences)

SEQ ID NO: 1 - DNA, 21 bp, Artificial Sequence
  siRNA against Mcl-1, sense sequence
ucggacucaaccucuacugtt

SEQ ID NO: 2 - DNA, 21 bp, Artificial Sequence
  siRNA against Mcl-1, antisense sequence
caguagagguugaguccgatt

SEQ ID NO: 3 - DNA, 21 bp, Artificial Sequence
  siRNA against Mcl-1, sense sequence
aucguugucucgagugaugtt

SEQ ID NO: 4 - DNA, 21 bp, Artificial Sequence
  siRNA against Mcl-1, antisense sequence
caucacucgagacaacgautt

SEQ ID NO: 5 - DNA, 21 bp, Artificial Sequence
  siRNA against Mcl-1, sense sequence
acaaagaggcugggaugggtt

SEQ ID NO: 6 - DNA, 21 bp, Artificial Sequence
  siRNA against Mcl-1, antisense sequence
cccaucccagccucuuugutt

SEQ ID NO: 7 - DNA, 21 bp, Artificial Sequence
  siRNA against luciferase GL2 (forward)
cguacgcggaauacuucgatt

SEQ ID NO: 8 - DNA, 21 bp, Artificial Sequence
  siRNA against luciferase GL2 (reverse)
ucgaaguauuccgcguacgtt

SEQ ID NO: 9 - DNA, 20 bp, Artificial Sequence
  primer for Mcl-1 PCR
agctgcatcgaaccattagc

SEQ ID NO: 10 - DNA, 20 bp, Artificial Sequence
  primer for Mcl-1 PCR
gctcctactccagcaacacc

(19) United States
(12) Patent Application Publication
(10) Pub. No.: US 2011/0160282 A1
(43) Pub. Date: Jun. 30, 2011

(54) NUCLEIC-ACID PHARMACEUTICAL COMPOSITION FOR CANCER THERAPY

(75) Inventors: Yu-Kyoung Oh, Seoul (KR); Gayong Shim, Yongin-si (KR); Sang-Hee Kim, Seoul (KR)

(73) Assignees: Korea University Research and Business Foundation, Seoul (KR); SNU R&DB Foundation, Seoul (KR)

(21) Appl. No.: 12/735,809

(22) PCT Filed: Feb. 18, 2009
(86) PCT No.: PCT/KR2009/000762

§ 371 Date: Jan. 12, 2011

(30) Foreign Application Priority Data
Feb. 19, 2008 (KR) 10-2008-0014724

(51) Int. Cl. A61K 31/713; C07H 21/00; A61P 35/00

(52) U.S. Cl. 514/44 A; 536/24.5

(57) ABSTRACT

The present invention provides at least one siRNA (small interfering RNA) which inhibits the expression of Mcl-1 within the cell, and which is selected from siRNA having SEQ. ID. NO. 1 sense sequence and SEQ. ID. NO. 2 antisense sequence, siRNA having SEQ. ID. NO. 3 sense sequence and SEQ. ID. NO. 4 antisense sequence, and siRNA having SEQ. ID. NO. 5 sense sequence and SEQ. ID. NO. 6 antisense sequence. The invention also provides a nucleic-acid pharmaceutical composition for cancer therapy comprising the same. The siRNA of the present invention kills cancer cells by inhibiting the expression of Mcl-1 which is commonly expressed in cancer cells by means of RNA-mediated interference (RNAi), and the composition of the present invention can be used as an outstanding anti-cancer preparation.

### TECHNICAL FIELD

The present invention relates to an siRNA (small interfering RNA) inhibiting the expression of Mcl-1 in cells, and a nucleic-acid pharmaceutical composition for treating cancer comprising the same.

### BACKGROUND ART

Proteins belonging to the Bcl-2 family are variously involved in the mitochondria-dependent apoptosis pathway. Bcl-2 and Bcl-xL proteins belonging to the Bcl-2 family are potent anti-apoptotic proteins, whereas Bax, Bak, Bid, Bad, etc. belonging to the Bcl-2 family are apoptosis-inducing proteins (Adams et al., Science, 281:1322, 1998). Therapeutic methods that target anti-apoptotic proteins such as Bcl-2 or Bcl-xL in order to increase the sensitivity of cancer cells to anticancer drugs and to inhibit the resistance of cancer cells to anticancer drugs are being actively studied, and a Bcl-2 antisense oligomer as an anticancer agent is now in clinical trials. These anti-apoptotic Bcl-2 family proteins function to interfere with apoptosis by inhibiting the release of cytochrome c from mitochondria.

Mcl-1 that is a protein belonging to the Bcl-2 family was first discovered in myeloid leukemia cells in the year 1993 (Michels, J., et al., 37:267, 2005). It was thought that the inhibition of Mcl-1 would induce the expression of pro-apoptotic proteins to destroy the mitochondrial membrane, thereby stimulating the release of cytochrome C, but an accurate pathway of apoptotic inhibition has not been found (Hussain, S. R., et al., Clin Cancer Res 13:2144, 2007).

However, a study supporting this thought was recently reported which indicates that Bim inhibits the activation of apoptosis-inducing proteins such as Bax and Bak, because Mcl-1 binds to Bim protein. Thus, it can be seen that, when Mcl-1 is inhibited, apoptosis is induced (Anthony L., et al., Molecular Cell 21:728, 2006).

Mcl-1 plays an essential role in embryo generation, lymphocyte maintenance, cell survival and the like (Edwards, S. W., et al., Biochemical Society Transactions 32:489, 2004). Mcl-1 is found in a wide range of cells and tissues, including adult cells, embryo tissues and epithelial tissues, and is induced by cytokines or growth factors. Several studies have been reported which indicate that Mcl-1 is inhibited in apoptotic cells and plays an important role as an oncogene in cell survival and tumorigenesis. Mcl-1 has been recognized as a new target for anticancer therapy, and many studies thereon have been conducted (Le Gouill, S., et al., 3:1259, 2004).

RNA-mediated interference (RNAi) is a phenomenon wherein a 21-25 nucleotide-long double stranded siRNA specifically binds to a transcript (mRNA transcript) having a complementary sequence and degrades the corresponding transcript to thereby inhibit the expression of a target protein of interest. As the RNA-mediated interference has recently suggested the solution to the problems encountered in the development of conventional chemical synthetic drugs, many studies have been conducted on the use of the RNA-mediated interference in the development of various therapeutic agents, particularly antitumor agents, which selectively inhibit the expression of a certain protein at the transcript level. Production of small-molecule chemical drugs takes a long development period of time and tremendous development costs until they are optimized to certain protein targets, whereas the most pronounced advantage of siRNA drugs using the RNA-mediated interference phenomenon is that it readily enables development of the optimized lead compounds for all the protein targets including non-druggable target substances. Protein or antibody drugs suffer from difficulties of production thereof due to complicated manufacturing processes, whereas siRNAs have significant advantages, including ease of synthesis, separation and purification, consequently relatively easy and convenient commercial-scale production, higher storage stability attributed to intrinsic nature of nucleic acid materials, as compared to protein drugs, and the like. Furthermore, siRNA-based drugs are receiving a great deal of attention as a new drug candidate, based on a variety of strengths such as specific molecular target-directed antagonism, unlike conventional drugs (David et al., Nature Chemical Biology, 2:711-719, 2006).

The primary challenge associated with siRNA-based therapy is the selection of the optimum sequence where siRNA has the highest activity in the target base sequence. It is known that the efficiency of RNA-mediated interference is significantly affected by a specific binding site for the target transcript. Based on the database accumulated for the past several years, algorithms have been developed which are capable of designing a sequence position of siRNA substantially inhibiting expression of the target RNA, instead of simply binding to the transcript, and are currently available to users. However, it cannot be said that all of siRNAs determined by an in silico method using computer algorithms can effectively inhibit target RNAs in real cells and in vivo. Furthermore, it is known that even when requirements necessary for complementary binding of siRNA to the target transcript are satisfied, the stability and intracellular location of RNAs and proteins, the state of proteins implicated in RNA-mediated interference, and a variety of other unknown factors are implicated in the determination of efficiency of RNA-mediated interference (Derek et al., Annual Rev. Biomed. Eng., 8:377-402, 2006).

### DISCLOSURE

### Technical Problem

It is an object of the present invention to provide at least one siRNA (small interfering RNA) that complementarily binds to an Mcl-1 transcript (mRNA transcript) base sequence to inhibit the expression of Mcl-1 in cells, and a nucleic-acid pharmaceutical composition for treating cancer comprising the same.

### Technical Solution

The present invention provides a nucleic-acid pharmaceutical composition for treating cancer comprising at least one siRNA inhibiting the expression of Mcl-1 in cells, the siRNA being selected from among an siRNA having a sense sequence of SEQ ID NO: 1 and an antisense sequence of SEQ ID NO: 2, an siRNA having a sense sequence of SEQ ID NO: 3 and an antisense sequence of SEQ ID NO: 4, and an siRNA having a sense sequence of SEQ ID NO: 5 and an antisense sequence of SEQ ID NO: 6.

The nucleic-acid pharmaceutical composition for treating cancer in accordance with the present invention, which comprises an siRNA inhibiting the expression of Mcl-1, may comprise a single siRNA species such that the siRNA can bind only to a single sequence position of the transcript (mRNA) of Mcl-1. Alternatively, the pharmaceutical composition of the present invention may comprise two or more siRNAs such that one or more sequence positions of the Mcl-1 transcript can be targeted.

As used herein, the term “siRNA” is intended to encompass siRNAs that have been chemically modified to prevent their rapid degradation by nucleases in vivo. Those skilled in the art will appreciate that it is possible to synthesize and modify siRNAs as desired, using any conventional method known in the art (Andreas Henschel, Frank Buchholzl and Bianca Habermann (2004) DEQOR: a web-based tool for the design and quality control of siRNAs. Nucleic Acids Research 32 (Web Server Issue):W113-W120). siRNA has a double-stranded structure, and thus is relatively stable compared to single-stranded ribonucleic acid or antisense oligonucleotide. However, siRNA is rapidly degraded by nucleases in vivo, so it is possible to reduce the degradation rate of siRNA through chemical modification. Chemical modification of siRNA to secure that siRNA is chemically stable and resistant against rapid degradation may be carried out by any conventional method well known to those skilled in the art. The most conventional method used in chemical modification of siRNA is boranophosphate or phosphorothioate modification. These materials form stable linkage between nucleosides of siRNA, thereby conferring resistance to nucleic acid degradation. Even though it is resistant to nucleic acid degradation, the boranophosphate-modified ribonucleic acid is not synthesized by a chemical reaction and is synthesized only by incorporation of boranophosphate into ribonucleic acid via in vitro transcription. The boranophosphate modification is a relatively easy method, but has a disadvantage associated with difficulty of site-directed modification at a certain position. On the other hand, the phosphorothioate modification is advantageous for introduction of a sulfur element into a desired site, but excessive phosphothioation may result in problems associated with decreased efficiency, toxicity, and formation of a non-specific RNA-induced silencing complex (RISC). For these disadvantages of the above-mentioned two modification methods, it may be preferred to employ a method which provides nuclease resistance via introduction of chemical modification only into a terminal position of ribonucleic acid (the region beyond the 3′ end). In addition, chemical modification of a ribose ring is also known to enhance nuclease resistance. Particularly, modification at 2′-position of the ribose present in cells leads to stabilization of double-stranded RNA. However, the stability of double-stranded RNA molecules increases only when a methyl group is correctly introduced into the above-specified position of the ribose ring. Also, introduction of excessively large numbers of methyl groups may adversely result in loss of ribonucleic acid-mediated interference. Such chemical modifications may also be made to enhance the in vivo pharmacokinetic residence time and efficiency (Mark et al., Molecular Therapy, 13:644-670, 2006).

In addition to chemical modifications, a safe and efficient delivery system is still required to increase the intracellular delivery efficiency of siRNA. For this purpose, the siRNA of the present invention may be incorporated in the form of a complex with a nucleic acid delivery system into the nucleic-acid pharmaceutical composition for treating cancer.

The nucleic acid delivery system for intracellular delivery of nucleic acid materials may be broadly divided into a viral vector system and a non-viral vector system. The most widely used system is a viral vector system, because it exhibits a high delivery efficiency and a long in vivo residence time. Among a variety of viral vectors, a retroviral vector, an adenoviral vector, an adeno-associated viral vector, and the like are mainly employed. These viral vectors are efficient in intracellular delivery of ribonucleic acid, but suffer from various problems associated with safety concerns, such as difficulty of recombination of ribonucleic acid into a virus having in vivo activity, induction of immune responses, undesirable random insertion of ribonucleic acid into host chromosomes, and the like. On the other hand, the non-viral vector exhibits various advantages, such as low toxicity and immune response, feasibility of repeated administration, convenient formation of a complex with ribonucleic acid, and easy mass production, compared to the viral vector system. In addition, a conjugate of the non-viral vector with a ligand specific for diseased cells or tissues enables organ/cell-targeted delivery of nucleic acid. Examples of the non-viral vector that may be used in the present invention include various formulations such as liposomes, cationic polymers, micelles, emulsions, nanoparticles, and the like. The nucleic acid delivery system can significantly enhance delivery efficiency of the desired nucleic acid into animal cells and can deliver nucleic acids into any type of animal cells, depending upon the intended use of the nucleic acid.

In one embodiment of the present invention, the nucleic acid delivery system may be a cationic micelle, a cationic emulsion or a cationic liposome.

The cationic micelle, the cationic emulsion or the cationic liposome may include, but is not limited to, at least one cationic lipid selected from the group consisting of 1,2-dioleoyl-sn-glycero-3-ethylphosphocholine (EPOPC), 1-palmitoyl-2-oleoyl-sn-glycero-3-ethylphosphocholine (EPOPC), 1,2-dimyristoyl-sn-glycero-3-ethylphosphocholine (EDMPC), 1,2-distearoyl-sn-glycero-3-ethylphosphocholine (SPC), 1,2-dipalmitoyl-sn-glycero-3-ethylphosphocholine (EDPPC), 1,2-dioleoyl-3-trimethylammonium-propane (DOTAP), N-[1-(2,3-dioleyloxy)propyl]-N,N,N-trimethylammonium chloride (DOTMA), and 3β-[N—(N′,N′-dimethylaminoethane)-carbamoyl]cholesterol (DC-Cholesterol).

Also, the cationic micelle, the cationic emulsion or the cationic liposome may further include, but is not limited to, at least one auxiliary lipid selected from the group consisting of 1,2-diacyl-sn-glycero-3-phosphoethanolamine (DOPE), 1,2-diphytanoyl-sn-glycero-3-phosphoethanolamine (DPhPE), 1,2-dioleoyl-sn-glycero-3-phosphocholine (DOPC), 1,2-dioleoyl-sn-glycero-3-[phospho-L-serine] (DOPS), 1,2-dioleoyl-sn-glycero-3-ethylphosphocholine (DO-Ethyl-PC), cholesterol, etc.

In another embodiment of the present invention, the nucleic acid delivery system may be a cationic polymer.

The cationic polymer may be at least one polymer selected from the group consisting of, but not limited to, poly-L-lysine (PLL), poly-L-ornithine, poly-L-histidine, bis(3-aminopropyl) terminated polytetrahydrofuran (AT-PTHF), polyacrylamide (PA), poly(α-[4-aminobutyl]-L-glycolic acid (PAGA), poly(2-aminoethyl propylene phosphate (PPE-EA), cationic derivatives of cyclodextrin, poly(2-(dimethylamino)ethyl methacrylate) (pDMAEMA), poly(4-vinylpyridine) (P4VP), O,O′-Bis(2-aminopropyl)polypropylene glycol-block-polyethylene glycol-block-polypropylene glycol, poly-N-ethyl-4-vinylpyridinium tribromide (PVP), chitosan, cationic chitosan derivatives, polyamidoamine (PAMAM), fractured PAMAM, polyethyleneimine (PEI), and polyethyleneimine derivatives.

The nucleic acid delivery system of the present invention, which is in the form of a cationic micelle, cationic emulsion, liposome or cationic polymer formulation, is positively charged. Thus, due to the presence of positive charges of the nucleic acid delivery system and negative charges of the nucleic acid, a complex between the nucleic acid delivery system and the nucleic acid may be formed by electrostatic bonding, even when they are simply mixed.

Moreover, the pharmaceutical composition for treating cancer in accordance with the present invention may further comprise, in addition to the siRNA component inhibiting the expression of Mcl-1, one or more conventionally known anticancer chemotherapeutic agents, whereby it can achieve synergistic therapeutic effects. Examples of cancer chemotherapeutic agents that can be administered in combination with the inventive siRNA inhibiting the expression of Mcl-1 include cisplatin, carboplatin, oxaliplatin, doxorubicin, daunorubicin, epirubicin, idarubicin, mitoxantrone, valubicin, curcumin, gefitinib, erlotinib, irinotecan, topotecan, vinblastine, vincristine, docetaxel, paclitaxel, etc.

The composition of the present invention may further comprise at least one pharmaceutically acceptable carrier, in addition to siRNA or a complex of siRNA with the nucleic acid delivery system. Examples of suitable pharmaceutically acceptable carriers include water, saline, PBS (phosphate buffered saline), dextrin, glycerol, and ethanol. These carries may be used alone or in any combination thereof. In addition, the composition of the present invention may be formulated so as to provide rapid, sustained or delayed release of the active ingredient after administration.

The siRNA or siRNA/nucleic acid delivery system complex of the present invention may be introduced into cells to treat cancer. As can be seen in Examples below, when the siRNA or siRNA/nucleic acid delivery system complex of the present invention is introduced into cells, the expression of Mcl-1 that is involved in carcinogenesis will be inhibited, resulting in death of cancer cells.

Accordingly, the present invention provides the use of at least one siRNA, which inhibits the expression of Mcl-1 in cells, for the preparation of an anticancer drug, the siRNA being selected from among an siRNA having a sense sequence of SEQ ID NO: 1 and an antisense sequence of SEQ ID NO: 2, an siRNA having a sense sequence of SEQ ID NO: 3 and an antisense sequence of SEQ ID NO: 4, and an siRNA having a sense sequence of SEQ ID NO: 5 and an antisense sequence of SEQ ID NO: 6. The present invention also provides a method for treating cancer comprising introducing into a cell of a subject in need of such treatment a therapeutically effective amount of at least one siRNA inhibiting the expression of Mcl-1 in the cell, the siRNA being selected from among an siRNA having a sense sequence of SEQ ID NO: 1 and an antisense sequence of SEQ ID NO: 2, an siRNA having a sense sequence of SEQ ID NO: 3 and an antisense sequence of SEQ ID NO: 4, and an siRNA having a sense sequence of SEQ ID NO: 5 and an antisense sequence of SEQ ID NO: 6. In the present invention, cancer treatment includes the prevention and inhibition of cancer.

When a desired nucleic acid is introduced into cells in vivo or ex vivo by means of the nucleic-acid pharmaceutical composition for cancer treatment of the present invention, it will function to selectively the expression of the target protein Mcl-1 or to correct mutations of the target gene, thus making it possible to treat cancer caused by overexpression of Mcl-1.

As used herein, the term “therapeutically effective amount” of the siRNA or siRNA/nucleic acid delivery system complex refers to an amount that is required to exert anticancer effects. Thus, the therapeutically effective amount of the active ingredient may vary depending upon various factors, including the type of disease, the severity of disease, the type of nucleic acid to be administered, the form of the composition, the patient's age, weight, physical condition, sex and dietary habits, administration time and route, treatment duration, and drugs such as co-administered chemical anticancer drugs. For adults, the composition for cancer treatment is preferably administered at a dose of 0.001 mg/kg to 100 mg/kg once a day. The composition may be administered via various routes, including oral, intranasal, intraocular, topical, intravenous, transdermal and intramuscular routes.

In Examples below, siRNAs effective for inhibition of Mcl-1 expression were synthesized from the full-length transcript sequence of Mcl-1 and transfected into tumor cell lines using a delivery system such as a liposome, a cationic polymer, etc., and RNA-mediated expression interference was then analyzed at the transcript level by reverse transcription-polymerase chain reaction (RT-PCR). In addition, in order to evaluate the effect of inhibition of Mcl-1 expression on the growth of tumor cells, tetrazolium 3-(4,5-dimethylthiazol-2-yl)-2,5-diphenyl tetrazolium bromide (MTT) staining, lactate dehydrogenase (LDH) assay, Annexin V-FITC (fluorescein isothiocyanate)/PI (propidium iodide) staining and cell staining were carried out on the cell groups treated with the Mcl-1 expression-inhibiting siRNAs.

The results obtained in Examples below revealed that an siRNA having a sense sequence of SEQ ID NO: 1 and an antisense sequence of SEQ ID NO: 2, an siRNA having a sense sequence of SEQ ID NO: 3 and an antisense sequence of SEQ ID NO: 4, and an siRNA having a sense sequence of SEQ ID NO: 5 and an antisense sequence of SEQ ID NO: 6 all effectively inhibited the expression of Mcl-1.

Accordingly, the present invention also provides an siRNA inhibiting the expression of Mcl-1 and having a sense sequence of SEQ ID NO: 1 and an antisense sequence of SEQ ID NO: 2.

The present invention also provides an siRNA inhibiting the expression of Mcl-1 and having a sense sequence of SEQ ID NO: 3 and an antisense sequence of SEQ ID NO: 4.

The present invention also provides an siRNA inhibiting the expression of Mcl-1 and having a sense sequence of SEQ ID NO: 5 and an antisense sequence of SEQ ID NO: 6.

The nucleic-acid pharmaceutical composition of the present invention may further comprise at least one siRNA inhibiting the expression of cancer-related genes other than Mcl-1.

As the siRNA which is additionally included in the nucleic-acid composition of the present invention, any siRNA may be used, as long as it is an siRNA for a gene that causes cancer by overexpression. For example, the siRNA inhibiting the expression of genes other than Mcl-1 may be selected from the group consisting of siRNAs inhibiting the expression of Wnt-1, Hec1, Survivin, Livin, Bcl-2, XIAP, Mdm2, EGF, EGFR, VEGF, VEGFR, GASC1, IGF1R, Akt1, Grp78, STAT3, STAT5a, β-catenin, WISP1, or c-myc.

The inventive composition further comprising the siRNA inhibiting the expression of cancer-related genes other than Mcl-1 can more effectively treat cancer compared to the case in which a siRNA for a single gene is used and the case in which siRNAs for cancer-related genes that cause cancer by overexpression are prepared individually and used in combination.

The present invention also provides the use of the above-described inventive composition for the preparation of anticancer drugs.

The present invention also provides a method for treating cancer comprising introducing into a cell of a subject in need of such treatment the above-described composition of the present invention.

In the present invention, cancer treatment includes the prevention and inhibition of cancer.

### Advantageous Effects

The siRNA of the present invention kills cancer cells by inhibiting the expression of Mcl-1, which is commonly expressed in cancer cells, using RNA-mediated interference (RNAi). Thus, the composition of the present invention can be used as an excellent anticancer drug.

### DESCRIPTION OF DRAWINGS

FIG. 1 shows the results of evaluating Mcl-1 expression-inhibiting siRNA-mediated inhibitory effects on Mcl-1 transcript expression in the human hepatoma cell line Hep3B using reverse transcription-polymerase chain reaction (RT-PCR).

FIG. 2 shows the results of evaluating Mcl-1 expression-inhibiting siRNA-mediated apoptotic effects on cancer cells in a human tumor cell line using an MTT (tetrazolium 3-(4,5-dimethylthiazol-2-yl)-2,5-diphenyl tetrazolium bromide) staining method.

FIG. 3 shows the results of evaluating the apoptotic effects of Mcl-1 expression-inhibiting siRNAs in the human hepatoma cell line Hep3B using a lactate dehydrogenase (LDH) assay.

FIG. 4 shows the results of evaluating the apoptotic effects of Mcl-1 expression-inhibiting siRNAs in the human hepatoma cell line Hep3B using an annexin V-FITC/PI staining assay.

FIG. 5 shows the results of evaluating the apoptotic effects of Mcl-1 expression-inhibiting siRNAs in the human hepatoma cell line Hep3B by a cell staining assay using a crystal violet dye.

FIG. 6 shows the results of evaluating tumor cell-killing effects, mediated by a combination of an siRNA inhibiting Mcl-1 expression and an siRNA inhibiting Wnt-1 or Hec1 expression, in the human hepatoma cell line Hep3B using an MTT (tetrazolium 3-(4,5-dimethylthiazol-2-yl)-2,5-diphenyl tetrazolium bromide) staining method.

FIG. 7 shows the results of evaluating the apoptotic effects of chemically modified, Mcl-1 expression-inhibiting siRNAs in the human hepatoma cell line Hep3B using a lactate dehydrogenase (LDH) assay.

### MODE FOR INVENTION

Advantages and features of the present invention, and a method for achieving them will be clarified with reference to examples that will be described later. The present invention may, however, be embodied in different forms and should not be construed as limited to the examples set forth herein. Rather, these examples are provided so that the disclosure of the present invention will be thorough and complete, and will fully convey the scope of the present invention to those skilled in the art. The scope of the present invention should be defined by the appended claims.

### Example 1

### Preparation of siRNAs Inhibiting the Expression of Mcl-1

siRNAs inhibiting the expression of Mcl-1 were synthesized and prepared using a Silencer siRNA Construction kit purchased from Ambion Inc. (Texas, USA) in accordance with the manufacturer's instruction. The sequences of the siRNAs are shown in Table 1 below.

TABLE 1

| | | Sense sequence (5′-3′) |
|---|---|---|
| Examples | SEQ ID NO | Antisense sequence (5′-3′) |
| 1-1 | 1 | UCGGACUCAACCUCUACUGTT |
| | 2 | CAGUAGAGGUUGAGUCCGATT |
| 1-2 | 3 | AUCGUUGUCUCGAGUGAUGTT |
| | 4 | CAUCACUCGAGACAACGAUTT |
| 1-3 | 5 | ACAAAGAGGCUGGGAUGGGTT |
| | 6 | CCCAUCCCAGCCUCUUUGUTT |

### Example 2

### Preparation Of Mcl-1 Expression-Inhibiting siRNA/Cationic Liposome Complexes

A complex of each of the Mcl-1 expression-inhibiting siRNAs prepared in Example 1 with a cationic liposome for delivering the siRNA was prepared.

First, the cell-fusogenic phospholipid 1,2-diacyl-sn-glycero-3-phosphoethanolamine (DOPE), cholesterol, and the cationic phospholipid 1,2-dioleoyl-sn-glycero-3-W ethylphosphocholine (EDOPC) (Avanti Polar Lipids Inc., USA) were taken in a molar ratio of 1:1:1, mixed in a glass vial, and then rotary-evaporated at a low speed in a nitrogen atmosphere until chloroform was completely evaporated, thereby preparing a lipid thin film. For preparation of multilamellar vesicles, 1 mL of a phosphate-buffered solution (PBS) was added to the above-prepared thin film, and the vial was then sealed, followed by vortexing for 3 min at 37° C. To obtain a uniform particle size, the film solution was passed three times through a 0.2-μm polycarbonate membrane using an extruder (Northern Lipids Inc., Canada), thus preparing a cationic liposome. The resulting cationic liposome was mixed with each of the Mcl-1 expression-inhibiting siRNAs prepared in Example 1, and the mixtures, were allowed to stand at room temperature for 20 min, thus preparing siRNA/cationic liposome complexes. The compositions of the siRNA/cationic liposome complexes prepared in Example 2 are summarized in Table 2 below.

TABLE 2
Compositions of siRNA/cationic liposome
complexes prepared in Example 2

| Example | Mcl-1 siRNA | Cationic liposome |
|---|---|---|
| 2-1 | Example 1-1 | DOPE + cholesterol + EDOPC |
| 2-2 | Example 1-2 | DOPE + cholesterol + EDOPC |
| 2-3 | Example 1-3 | DOPE + cholesterol + EDOPC |

### Example 3

### Preparation of Mcl-1 Expression-Inhibiting siRNA/Cationic Polymer Complexes

Each of the Mcl-1 expression-inhibiting siRNAs (Example 1-1 and Example 1-2) prepared in Example 1 was mixed with the cationic polymer polyethylenimine (PEI) to prepare complexes. Specifically, 25-kD polyethylenimine (PEI) (Sigma-Aldrich, USA) was dissolved in water, adjusted to a molar concentration of 1 mM, placed in a 100-mL Pyrex glass round-bottom flask, and adjusted to a pH of 5 by addition of 1N hydrochloric acid. Then, the solution was passed through a 0.2-μm pore-size syringe filter membrane to remove impurities, thereby preparing a cationic polymer. Among the Mcl-1 expression-inhibiting siRNAs prepared in Example 1, siRNAs of Example 1-1 and Example 1-2 were selected. The above-prepared cationic polymer was mixed with each of the selected siRNAs, and the mixtures were allowed to stand at room temperature for 20 min to prepare complexes.

### Comparative Example 1

### Preparation of Luciferase GL2 Expression-Inhibiting siRNA/Cationic Liposome Complex

As a negative control for comparison of cytotoxicity of siRNA per se, a commercially available siRNA inhibiting the expression of luciferase GL2 was purchased from Samchully Pharmaceuticals Co., Ltd. (Seoul, Korea). The base sequence of the luciferase GL2 expression-inhibiting siRNA has 5′-CGUACGCGGAAUACUUCGATT-3′ (forward) and 5′-UCGAAGUAUUCCGCGUACGTT-3′) (reverse). The luciferase GL2 expression-inhibiting siRNA was mixed with the cationic liposome prepared in Example 2, and the mixture was allowed to stand at room temperature for 20 min, thereby preparing a complex of the siRNA with the cationic liposome.

<Culture of Hepatoma Cell Line Hep3B, Lung Cancer Cell Line A549, and Melanoma Cell Lines A375 and WM2664>

The hepatoma cell line Hep3B and the lung cancer cell line A549 were purchased from American Type Culture Collection (ATCC, USA). The melanoma cell lines A375 and WM2644 were purchased from the Korean Cell Line Bank (KCLB). Each of the cell lines was cultured in minimal essential medium (MEM, Gibco, USA) containing 10% w/v fetal bovine serum (FBS, HyClone Laboratories Inc., USA) and 100 units/mL of penicillin or 100 μg/mL of streptomycin.

### Example 4

### Evaluation of Inhibitory Effects of Mcl-1 Expression-Inhibiting siRNAs on Expression of Mcl-1 Transcript Using Reverse Transcription-Polymerase Chain Reaction (RT-PCR)

In order to evaluate the apoptotic effects of Mcl-1 expression-inhibiting siRNA-containing compositions on cancer cells, experiments were carried out according to the following procedure using reverse transcription-polymerase chain reaction (RT-PCR).

On the day prior to the experiment, Hep3B cells were seeded on 24-well plates at a density of 8×10⁴ cells/well.

When cells of each plate were grown to a confluence of about 50-70%, the culture media were replaced with 250 μl/well of fresh media.

50 μl of serum-free medium was added to each Eppendorf tube to which each of the luciferase GL2 expression-inhibiting siRNA/cationic liposome complex composition of Comparative Example 1 and three Mcl-1 expression-inhibiting siRNA/cationic liposome complex compositions of Examples 2-1 to 2-3 was then added. The final concentration of siRNA in the media was adjusted to 50 nM. These materials were slowly pipetted, mixed and allowed to stand at room temperature for 20 min, thus preparing complexes. Each of the prepared complexes and the cationic liposome alone prepared in Example 2 was added to the well plate, followed by cell culture in a CO₂ incubator at 37° C. for 24 hours.

After 24 hours, total RNA was isolated from the cultured cells using Trizol reagent (Invitrogen, Carlsbad, Calif., USA) and then reverse-transcribed into cDNA using AccuPower RT PreMix (Bioneer, Daejeon, Korea). The Mcl-1-specific primer used for PCR had a sequence of 5′-AGCTGCATCGAACCATTAGC-3′ (left) and a sequence of 5′-GCTCCTACTCCAGCAACACC-3′ (right), and the size of the PCR product was 176 bp in length. The expression level of the Mcl-1 transcript (mRNA transcript) was quantitatively determined by normalizing the band density of the Mcl-1-specific PCR product to a band density obtained by amplification of a GAPDH (glyceraldehyde-3-phosphate dehydrogenase) transcript.

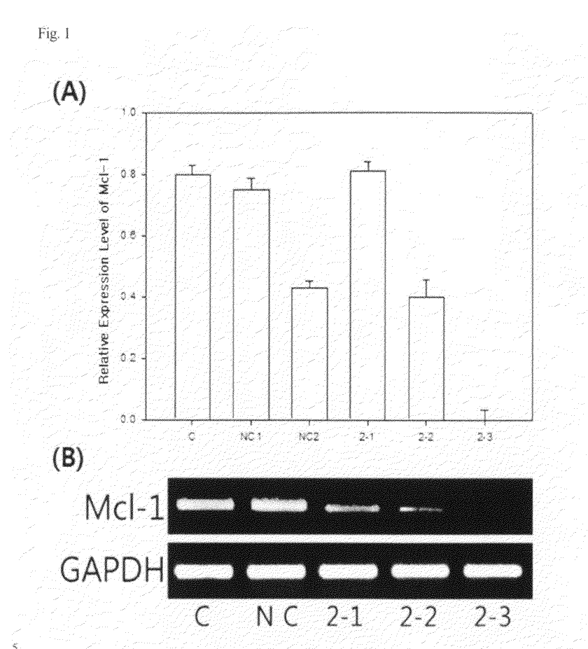

FIG. 1 shows the results of examining Mcl-1 expression-inhibiting siRNA-mediated inhibitory effects on Mcl-1 transcript expression in the human hepatoma cell line Hep3B using RT-PCR. FIG. 1A is a numerical representation of the relative expression of Mcl-1 transcript, and FIG. 1B is a representative electrophoretic pattern showing the expression levels of Mcl-1 transcript. In FIG. 1, “C” represents a control group, “NC1” represents a group treated with the cationic liposome alone prepared in Example 2, “NC2” represents a group treated with the complex of Comparative Example 1, and “2-1”, “2-2” and “2-3” represent groups treated with the complexes of Examples 2-1 to 2-3, respectively. The control group (C) was a non-treated group where expression of the Mcl-1 transcript was observed; the group (NC1) treated with the cationic liposome alone, and the group (NC2) treated with the complex of Comparative Example 1 were luciferase GL2 RNA-treated groups where there was no change in expression of the Mcl-1 transcript, as compared to the control group; and the groups treated with the complexes of Example 2-1 to 2-3, respectively, exhibited a significant decrease in the expression of the Mcl-1 transcript, compared to the control group. Therefore, it can be seen from FIG. 1 that the siRNAs prepared in Examples 1-1 to 1-3 were delivered into the Hep3B cells, thereby selectively inhibiting the expression of Mcl-1.

### Example 5

### Evaluation of Antitumor Effects of Mcl-1 Expression-Inhibiting siRNAs Using MTT Assay

In order to evaluate the apoptotic effects of Mcl-1 expression-inhibiting siRNA-containing compositions on cancer cells, experiments were carried out on a hepatoma cell line, a melanoma cell line and a lung cancer cell line according to the following procedure using an MTT (3-(4,5-dimethylthiazol-2-yl)-2,5-diphenyl tetrazolium bromide) staining method.

5-1: Evaluation of Antitumor Effects on Hepatoma Cell Line

On the day prior to the experiment, hepatoma Hep3B cells were seeded on 24-well plates at a density of 8×10⁴ cells/well. When cells of each plate were grown to a confluence of about 50-70%, the culture media of the plates were replaced with 250 μl/well of fresh serum-free media.

50 μl of serum-free medium was added to each Eppendorf tubes to which each of the luciferase GL2 expression-inhibiting siRNA/cationic liposome complex composition of Comparative Example 1 and the Mcl-1 expression-inhibiting siRNA/cationic liposome complex compositions of Examples 2-1 to 2-3 was then added. The final concentration of siRNA in the media was adjusted to 50 nM. These materials were slowly pipetted, mixed and allowed to stand at room temperature for 20 min, thus preparing complexes. Each of the prepared complexes and the cationic liposome alone prepared in Example 2 was added to the well plate, followed by cell culture in a CO₂ incubator at 37° C. for 24 hours.

48 hours after treatment of the cells with each of the complex compositions, an MTT solution was added to make 10% of the medium, followed by cell culture for another 4 hours. Then, the supernatant was discarded and a 0.06 N isopropanol hydrochloride solution was added to the medium. The absorbance of the medium was then measured at 570 nm using an ELISA reader. Non-treated cells were used as a control group.

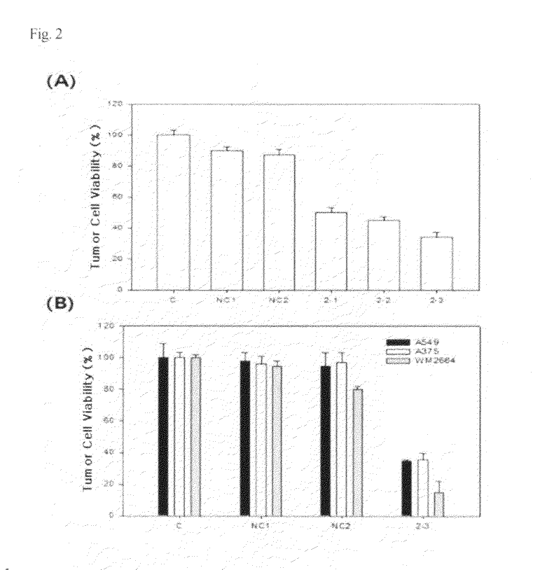

FIG. 2 shows the results of examining Mcl-1 expression-inhibiting siRNA-mediated tumor cell killing effects using the MTT (tetrazolium 3-(4,5-dimethylthiazol-2-yl)-2,5-diphenyl tetrazolium bromide) staining method. FIG. 2A shows the results of examining siRNA-mediated tumor cell killing effects in the human hepatoma cell line Hep3B. In FIG. 2A, “C” represents a control group, “NC1” represents a group treated with the cationic liposome alone prepared in Example 2, “NC2” represents a group treated with the complex of Comparative Example 1, and “2-1”, “2-2” and “2-3” represent groups treated with the complexes of Examples 2-1 to 2-3, respectively. The control group (C) was a non-treated group where the tumor cell viability should be 100%; the group (NC1) treated with the delivery system liposome alone exhibited no significant changes in the cell viability, compared to the control group, because it contained no siRNA; the group (NC2) treated with the complex of Comparative Example 1 was a group treated with luciferase GL2-inhibiting siRNA which exhibited substantially no effects on the viability of tumor cells; and the groups treated with the complexes of Examples 2-1 to 2-3, respectively, exhibited a decrease in the viability of tumor cells, compared to the control group. Therefore, it can be seen from FIG. 2 that the siRNAs prepared in Examples 1-1 to 1-3 were delivered into the Hep3B cells, thereby selectively inhibiting the expression of Mcl-1, suggesting that these siRNAs exerted antitumor effects.

5-2: Evaluation of Antitumor Effects on Lung Cancer Cell Line and Melanoma Cell Line

This Example was carried out in the same manner as Example 5-1, except that the lung cancer line A549 and the melanoma cell lines A375 and WM2664 were used instead of the Hep3B cell line.

FIG. 2 shows the results of examining Mcl-1 expression-inhibiting siRNA-mediated tumor cell killing effects using the MTT (tetrazolium 3-(4,5-dimethylthiazol-2-yl)-2,5-diphenyl tetrazolium bromide) staining method. FIG. 2B shows the results of examining siRNA-mediated tumor cell killing effects in the human lung cancer line A549 and the melanoma cell lines A375 and WM2664. The control group (C) was a non-treated group where the tumor cell viability should be 100%; the group (NC1) treated with the delivery system liposome alone exhibited no significant changes in the cell viability, compared to the control group, because it contained no siRNA; the group (NC2) treated with the complex of Comparative Example 1 was a group treated with luciferase GL2-inhibiting siRNA; and the group (2-3) treated with the Mcl expression-inhibiting siRNA of Example 2-3 exhibited a decrease in the viability of tumor cells, compared to the control group. Therefore, it can be seen from FIG. 2B that the siRNA prepared in Example 1-3 was delivered into the A549, A375 and WM2664 cells, thereby selectively inhibiting the expression of Mcl-1, suggesting that the siRNA exerted antitumor effects.

### Example 6

### Evaluation of Antitumor Effects of Mcl-1 Expression-Inhibiting siRNA Using Lactate Dehydrogenase (LDH) Assay

In order to evaluate the effects of Mcl-1 expression-inhibiting siRNA-containing compositions on damage to tumor cells, experiments were carried out according to the following procedure using an LDH Cytotoxicity Detection kit (TAKARA Bio Inc., Otsu Shiga, Japan) that detects lactate dehydrogenase (LDH), extracellularly secreted due to damage to the tumor cells, with high sensitivity.

On the day prior to the experiment, Hep3B cells were seeded on 24-well plates at a density of 8×10⁴ cells/well. When cells of each plate were grown to a confluence of about 50-70%, the culture media were replaced with 250 μl/well of fresh serum-free media.

50 μl of serum-free medium was added to each Eppendorf tube to which each of the luciferase GL2 expression-inhibiting siRNA/cationic liposome complex composition of Comparative Example 1 and the Mcl-1 expression-inhibiting siRNA/cationic liposome complex compositions of Examples 2-1 to 2-3 was then added. The final concentration of siRNA in the media was adjusted to 50 nM. These materials were slowly pipetted, mixed and allowed to stand at room temperature for 20 min, thus preparing complexes. Each of the prepared complexes and the cationic liposome alone prepared in Example 2 was added to the well plate, followed by cell culture in a CO₂ incubator at 37° C. At this time, Triton X-100 was added at a concentration of 3% to the plate such that the maximum LDH activity could be measured, followed by cell culture in a CO₂ incubator at 37° C. 48 hours after treatment of the cells with each of the complexes, the tissue culture plate was centrifuged at 250×g for 10 min, and then 100 μl/well of the supernatant was transferred to another clear 96-well plate. A reaction mixture was prepared according to the manufacturer's protocol, and 100 μl of the reaction mixture was added to each well. The plate was shielded from light and allowed to stand at room temperature for 30 min. The absorbance of the plate was measured at 492 nm using an ELISA reader. A blank medium was used as a negative control, and cells treated with 3% Triton X-100 were used as a positive control. Tumor cell damage (%) was calculated according to the following equation: tumor cell damage (%)=[(absorbance of experimental group-absorbance of negative control group)/(absorbance of positive control group-absorbance of negative control group)×100].

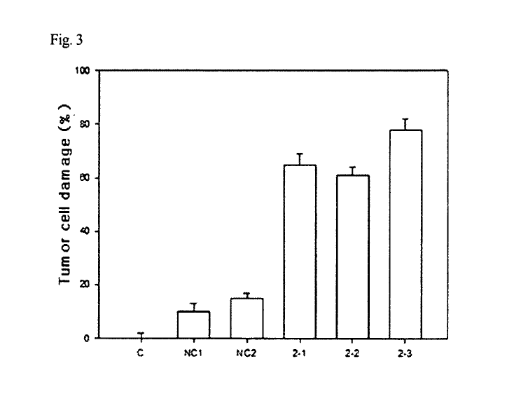

FIG. 3 shows the results of examining the apoptotic effects of Mcl expression-inhibiting siRNAs in the human hepatoma cell line Hep3B using the LDH assay. In FIG. 3, “C” represents a control group, “NC1” represents a group treated with the cationic liposome alone prepared in Example 2, “NC2” represents a group treated with the complex of Comparative Example 1, and “2-1”, “2-2” and “2-3” represent groups treated with the complexes of Examples 2-1 to 2-3, respectively. The control group “C” was a non-treated group where there were no changes in the tumor cell damage (%); the group “NC1” treated with the cationic liposome alone exhibited no significant changes in the tumor cell damage (%) compared to the control group, because it contained no siRNA; the group “NC2” treated with the complex of Comparative Example 1 exhibited no significant changes in the tumor cell damage (%), because the luciferase GL2-inhibiting siRNA did not cause RNA-mediated interference; and the groups treated with the complexes of Examples 2-1 to 2-3, respectively, exhibited an increase in the tumor cell damage (%) compared to the control group. Therefore, it can be seen from FIG. 3 that the siRNAs prepared in Examples 1-1 to 1-3 were delivered into the Hep3B cells, thereby selectively inhibiting the expression of Mcl-1, suggesting that these siRNAs exerted antitumor effects.

### Example 7

### Evaluation of Cancer Cell Killing Effects of Mcl-1 Expression-Inhibiting siRNAs Using FACS Assay

In order to evaluate the effects of Mcl-1 expression-inhibiting siRNA-containing compositions on the death of cancer cells, experiments were carried out according to the following procedure using a Fluorescence Activated Cell Sorting (FACS) assay.

The hepatoma cell line (Hep3B) was treated with each of the luciferase GL2 expression-inhibiting siRNA/cationic liposome complex of Comparative Example 1, the Mcl-1 expression-inhibiting siRNA/cationic liposome complex composition of Example 2-2, and the Mcl-1 expression-inhibiting siRNA/polyethyleneimine cationic polymer complex composition of Example 3, followed by evaluation of apoptosis. The evaluation of apoptosis was carried out using an Annexin V-FITC Apoptosis Detection kit (BD Biosciences, USA).

On the day prior to the experiment, Hep3B cells were seeded on 6-well plates at a density of 2×10⁵ cells/well. When cells of each plate were grown to a confluence of about 40-50%, the culture media of the plates were replaced with 1400 μl/well of fresh serum-free media.

50 μl of serum-free medium was added to each Eppendorf tube to which each of the luciferase GL2 expression-inhibiting siRNA/liposome complex composition of Comparative Example 1, the Mcl-1 expression-inhibiting siRNA/liposome complex composition of Example 2-2 and the Mcl-1 expression-inhibiting siRNA/polyethyleneimine cationic polymer complex composition of Example 3 was then added. The final concentration of siRNA in the media was adjusted to 50 nM. These materials were slowly pipetted, mixed and allowed to stand at room temperature for 20 min, thus preparing complexes. Each of the prepared complexes was added to the well plate, followed by cell culture in a CO₂ incubator at 37° C. 92 hours after treatment of the cells with each of the complexes, the cultured cells were collected and washed twice with PBS, followed by staining for 30 min under light shielding conditions, using annexin V-FITC and propidium iodide (PI) provided from the kit. Then, the apoptotic efficiency of the complex was analyzed by the shift of the fluorescence intensity peak using a BD FACS CALIBUR (BD Biosciences, USA). The shift of the fluorescence intensity peak was normalized using each of a cell group not treated with RNA, and cell groups treated with annexin V-FITC or PI alone and stained.

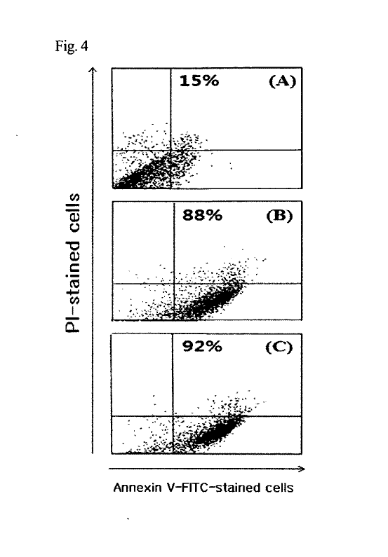

FIG. 4 shows the results of examining the apoptotic effects of Mcl-1 expression-inhibiting siRNAs in the human hepatoma cell line Hep3B using the annexin V-FITC/PI staining method. In FIG. 4, “A” represents a group treated with the complex of Comparative Example 1, “B” represents a group treated with the complex of Example 2-2, and “C” represents a group treated with the complex of Example 3. In the group treated with the complex composition of Comparative Example 1, the percentage of annexin V-positive cells, that is, cells that underwent apoptosis, was 15% (see FIG. 4A). On the other hand, in the group treated with the complex of Example 2-2, the percentage of annexin V-positive cells was 88%, suggesting that the percentage of cells that underwent apoptosis significantly increased (FIG. 4B). In the cell group treated with the complex of Example 3 containing two Mcl-1 expression-inhibiting siRNAs (siRNA of Example 1-1+siRNA of Example 1-2), the percentage of annexin V-positive cells that underwent apoptosis was increased to 92% (FIG. 4C). Therefore, it can be seen from FIG. 4 that the siRNA prepared in Example 1-2 was delivered into the Hep3B cells, thereby selectively inhibiting the expression of Mcl-1, suggesting that it had antitumor effects. In addition, as observed in the group treated with the complex of Example 3, the combined use of two effective siRNAs also exhibited significant antitumor effects.

### Example 8

### Evaluation of Cancer Cell Killing Effects of Mcl-1 siRNAs Using Staining of Residual Cells

In order to evaluate the apoptotic effects of Mcl-1 expression-inhibiting siRNA-containing compositions on cancer cells, experiments were carried out according to the following procedure using the staining of residual cells.

The hepatoma cell line (Hep3B) was treated with each of the luciferase GL2 expression-inhibiting siRNA/cationic liposome complex of Comparative Example 1 and the Mcl-1 expression-inhibiting siRNA/cationic liposome complex composition of Example 2-1 or Example 2-3, followed by evaluation of apoptosis. The evaluation of apoptosis was carried out by a cell staining method using a crystal violet dye.

On the day prior to the experiment, Hep3B cells were seeded on 6-well plates at a density of 2×10⁵ cells/well. When cells of each plate were grown to a confluence of about 40-50%, the culture media of the plates were replaced with 1400 μl/well of fresh serum-free media.

50 μl of serum-free medium was added to each Eppendorf tube to which each of the luciferase GL2 expression-inhibiting siRNA/liposome complex composition of Comparative Example 1 and the Mcl-1 expression-inhibiting siRNA/liposome of Example 2-1 or 2-3 was then added. The final concentration of siRNA in the media was adjusted to 50 nM. These materials were slowly pipetted, mixed and allowed to stand at room temperature for 20 min, thus preparing complexes. Each of the prepared complex was added to the well plate, followed by cell culture in a CO₂ incubator at 37° C. 92 hours after treatment of the cells with each complex, the cultured cells of the plate were washed with PBS, and stained for 1 min with 500 μl of a solution containing 0.5% crystal violet and 20% methanol, followed by removal of the solution. Then, cells remaining on the plate were observed.

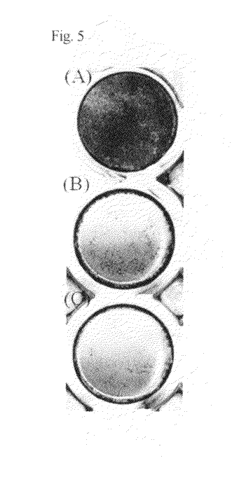

FIG. 5 shows the results of examining the apoptotic effects of Mcl-1 expression-inhibiting siRNAs in the human hepatoma cell line Hep3B by the cell staining method using a crystal violet dye. In FIG. 5, “A” represents a group treated with the complex of Comparative Example 1, “B” represents a group treated with the complex of Example 2-1, and “C” represents a group treated with the complex of Example 2-3. When compared with the group treated with the complex of Comparative Example 1 (FIG. 5A), the groups treated with the composition of Example 2-1 or the composition of Example 2-3 (FIGS. 5B and 5C), which exhibited the inhibition of Mcl-1 expression in Example 4, exhibited a decreased proportion of stained residual cells, because damaged tumor cells were detached from the well plate. Therefore, it can be seen from FIG. 5 that the siRNA/liposome complex compositions prepared in Example 2-1 and 2-3 exhibited selective inhibition of the Mcl-1 expression, suggesting that these complex compositions had antitumor effects.

### Example 9

### Evaluation of Antitumor Effects after Combined Treatment with siRNA Inhibiting Expression of Mcl-1 and siRNA Inhibiting Expression of Other Genes

The cancer cell killing effects of combined treatment with siRNAs inhibiting the expression of two or more genes were evaluated by treating cells with Mcl-1 expression-inhibiting siRNA in combination with another siRNA inducing cancer cell apoptosis. For this purpose, experiments were carried out according to the following procedure using the MTT (3-(4,5-dimethylthiazole-2-yl)-2,5-di-phenyl tetrazolium bromide) method.

On the day prior to the experiment, Hep3B cells were seeded on 24-well plates at a density of 8×10⁴ cells/well. When cells of each plate were grown to a confluence of about 50-70%, the culture media of the plates were replaced with 250 μl/well of fresh serum-free media.

50 μl of serum-free medium was added to each Eppendorf tube to which each of the luciferase GL2 expression-inhibiting siRNA/liposome complex composition of Comparative Example 1, the Mcl-1 expression-inhibiting siRNA/cationic liposome complex composition, which exhibited the best antitumor effect, a complex composition of the Mcl-1 expression-inhibiting siRNA of Example 1-3 and a Wnt-1 expression-inhibiting siRNA with a cationic liposome, and a complex composition of the Mcl-1 expression-inhibiting siRNA of Example 1-3 and a Hec1 expression-inhibiting siRNA with a cationic liposome was then added. The Wnt-1 (catalog number: M-003937-00) and Hec1 (catalog number: M-004106-00) expression-inhibiting siRNAs were purchased from DHARMACON (Lafayette, Co., USA).

The final concentration of siRNA in the media was adjusted to 50 nM. These materials were slowly pipetted, mixed and allowed to stand at room temperature for 20 min, thus preparing complexes. Each of the prepared complexes and the cationic liposome alone prepared in Example 2 was added to the well plate, followed by cell culture in a CO₂ incubator at 37° C. for 24 hours.

48 hours after treatment of the cells with each of the complexes, an MTT (3-(4,5-dimethylthiazole-2-yl)-2,5-di-phenyl tetrazolium bromide) solution was added to make 10% of the medium, followed by cell culture for another 4 hours. Then, the supernatant was discarded and a 0.06 N hydrochloric acid isopropanol solution was added to the medium. The absorbance of the medium was then measured at 570 nm using an ELISA reader.

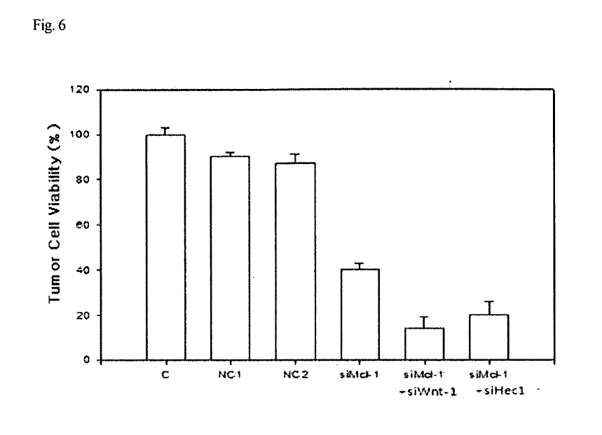

FIG. 6 shows the results of examining tumor cell killing effects, mediated by combined treatment with Mcl-1 expression-inhibiting siRNA and Wnt-1 expression-inhibiting siRNA or Hec1 expression-inhibiting siRNA, in the human hepatoma cell line Hep3B using the MTT (3-(4,5-dimethylthiazol-2-yl)-2,5-diphenyl tetrazolium bromide) staining method. In FIG. 6, “C” represents a control group, “NC1” represents a group treated with the cationic liposome alone prepared in Example 2, “NC2” represents a group treated with the complex of Comparative Example 1, siMcl-1 represents a group treated with the siRNA/cationic liposome complex of Example 2-3, and siMcl-1+siWnt-1 and siMcl-1+siHec1 represent groups treated with the complex of Mcl-1 expression-inhibiting siRNA of Example 1-3 and Wnt-1 or Hec1 with the cationic liposome. The control group “C” was a non-treated group where the tumor cell viability should be 100%; the group “NC1” treated with the delivery system liposome alone exhibited no significant changes in the cell viability compared to the control group, because it contained no siRNA; the group “NC2” treated with the complex of Comparative Example 1 was a group treated with luciferase GL2-inhibiting siRNA which exhibited no effects on the viability of tumor cells; and the groups treated with “siMcl-1+siWnt-1” and “siMcl-1+siHec1” (compositions containing, in addition to Mcl-1 expression-inhibiting siRNA of Example 1-3, Wnt-1 or Hec1 expression-inhibition siRNA) exhibited a decrease in the viability of tumor cells compared to the group treated with “siMcl-1” (siRNA/cationic liposome complex of Example 2-3). Therefore, it can be seen from FIG. 6 that the composition containing, in addition to Mcl-1 expression-inhibiting siRNA, cancer cell apoptosis-inducing siRNA such as Wnt-1 or Hec1 expression-inhibiting siRNA, was delivered into the Hep3 cells, thereby providing selective inhibition of genes targeted by the respective siRNAs, suggesting that the composition exhibited higher antitumor effects compared to the case of treatment with the Mcl-1 expression-inhibiting siRNA alone.

### Example 10

### Preparation of Complex of Chemically Modified, Mcl-1 Expression-Inhibiting siRNA with Cationic Liposome

The region beyond the 3′ end of the Mcl-1 expression-inhibiting siRNAs of Example 1 was chemically modified with phosphorothioate, and complexes of the chemically modified, Mcl-1 expression-inhibiting siRNAs with a cationic liposome delivering the siRNAs were prepared.

The chemical modification of the Mcl-1 expression-inhibiting siRNAs was carried out by Samchully Pharmaceuticals Co., Ltd. (Seoul, Korea).

To prepare a cationic liposome, the cell-fusogenic phospholipid 1,2-diacyl-sn-glycero-3-phosphoethanolamine (DOPE), cholesterol, and the cationic phospholipid 1,2-dioleoyl-sn-glycero-3-ethylphosphocholine (EDOPC) (Avanti Polar Lipids Inc., USA) were taken in a molar ratio of 1:1:1, mixed in a glass vial, and then rotary-evaporated at a low speed in a nitrogen atmosphere until chloroform was completely evaporated, thereby preparing a lipid thin film. For preparation of multilamellar vesicles, 1 mL of a phosphate-buffered solution (PBS) was added to the above-prepared thin film, and the vial was then sealed, followed by vortexing for 3 min at 37° C. To obtain a uniform particle size, the film solution was passed three times through a 0.2-μm polycarbonate membrane using an extruder (Northern Lipids Inc., Canada), thus preparing a cationic liposome. The resulting cationic liposome was mixed with each of the Mcl-1 expression-inhibiting siRNAs that have been modified with phosphorothioate at the region beyond the 3′ end. The mixtures were allowed to stand at room temperature for 20 min, thus preparing siRNA/cationic liposome complexes. The compositions of the phosphorothioate-modified siRNA/cationic liposome complexes prepared in Example 10 are summarized in Table 3 below.

TABLE 3
Compositions of the phosphorothioate-modified siRNA/
cationic liposome complexes prepared in Example 10

| Example | GASC1 siRNA | Cationic liposome |
|---|---|---|
| 10-1 | siRNA of Example 1-1 | DOPE + cholesterol + EDOPC |
| | modified with | |
| | phosphorothioate | |
| 10-2 | siRNA of Example 1-2 | DOPE + cholesterol + EDOPC |
| | modified with | |
| | phosphorothioate | |
| 10-3 | siRNA of Example 1-3 | DOPE + cholesterol + EDOPC |
| | modified with | |
| | phosphorothioate | |

### Example 11

### Evaluation of Antitumor Effects of Chemically Modified, Mcl-1 Expression-Inhibiting siRNAs Using Lactate Dehydrogenase (LDH) Assay

In order to evaluate the effects of chemically modified, Mcl-1 expression-inhibiting siRNA-containing compositions on damage to tumor cells, experiments were carried out according to the following procedure using an LDH Cytotoxicity Detection kit (TAKARA Bio Inc., Otsu Shiga, Japan) that detects lactate dehydrogenase (LDH), extracellularly secreted due to damage to the tumor cells, with high sensitivity.

On the day prior to the experiment, Hep3B cells were seeded on 24-well plates at a density of 8×10⁴ cells/well. When cells of each plate were grown to a confluence of about 50-70%, the culture media were replaced with 250 μl/well of fresh serum-free media.

50 μl of serum-free medium was added to each Eppendorf tube to which each of the luciferase GL2 expression-inhibiting siRNA/cationic liposome complex composition of Comparative Example 1, and the Mcl-1 expression-inhibiting siRNA/cationic liposome complex compositions of Examples 10-1 to 10-3, which have been modified with phosphorothioate at the 3′ end, was then added. The final concentration of siRNA in the media was adjusted to 50 nM. These materials were slowly pipetted, mixed and allowed to stand at room temperature for 20 min, thus preparing complexes. Each of the prepared complexes and the cationic'liposome alone prepared in Example 2 was added to the well plate, followed by cell culture in a CO₂ incubator at 37° C. At this time, Triton X-100 was added at a concentration of 3% to the plate such that the maximum LDH activity could be measured, followed by cell culture in a CO₂ incubator at 37° C. 48 hours after treatment of the cells with each of the complexes, the tissue culture plate was centrifuged at 250×g for 10 min, and then 100 μl/well of the supernatant was transferred to another transparent 96-well plate. A reaction mixture was prepared according to the manufacturer's protocol, and 100 μl of the reaction mixture was added to each well. The plate was shielded from light and allowed to stand at room temperature for 30 min. The absorbance of the plate was measured at 492 nm using an ELISA reader. A blank medium was used as a negative control, and cells treated with 3% Triton X-100 were used as a positive control. Tumor cell damage (%) was calculated according to the following equation: tumor cell damage (%)=[(absorbance of experimental group-absorbance of negative control group)/(absorbance of positive control group-absorbance of negative control group)×100].

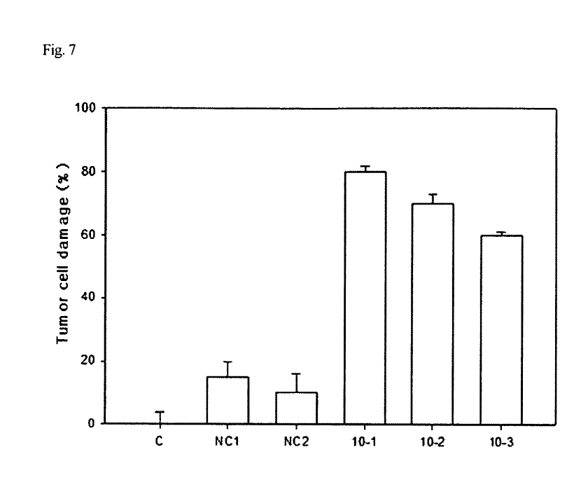

FIG. 7 shows the results of examining the apoptotic effects of Mcl expression-inhibiting siRNAs in the human hepatoma cell line Hep3B using the LDH assay. In FIG. 7, “C” represents a control group, “NC1” represents a group treated with the cationic liposome alone prepared in Example 2, “NC2” represents a group treated with the complex of Comparative Example 1, and “10-1”, “10-2” and “10-3” represent groups treated with the complexes of Examples 10-1 to 10-3, respectively. The control group “C” was a non-treated group where there were no changes in the tumor cell damage (%); the group “NC1” treated with the cationic liposome alone exhibited no significant changes in the tumor cell damage (%) compared to the control group, because it contained no siRNA; the group “NC2” treated with the complex of Comparative Example 1 exhibited no significant changes in the tumor cell damage (%), because the luciferase GL2-inhibiting siRNA did not cause RNA-mediated interference; and the groups treated with the complexes of Examples 10-1 to 10-3, respectively, exhibited an increase in the tumor cell damage (%) compared to the control group. Therefore, it can be seen from FIG. 7 that the chemically modified, Mcl-1 expression-inhibiting siRNAs prepared in Examples 10-1 to 10-3 were delivered into the Hep3B cells, thereby selectively inhibiting the expression of Mcl-1, suggesting that these siRNAs exerted antitumor effects.

### INDUSTRIAL APPLICABILITY

As described above, the present invention a siRNA (small interfering RNA) inhibiting the expression of Mcl-1 in cells, and a nucleic-acid pharmaceutical composition comprising the same. The siRNA of the present invention kills cancer cells by inhibiting the expression of Mcl-1, which is commonly expressed in cancer cells, using RNA-mediated interference (RNAi). Thus, the composition of the present invention can be used as an excellent anticancer drug.

1. A nucleic-acid pharmaceutical composition for treating cancer comprising at least one siRNA inhibiting expression of Mcl-1 in cells, the siRNA being selected from the group consisting of an siRNA having a sense sequence of SEQ ID NO: 1 and an antisense sequence of SEQ ID NO: 2; an siRNA having a sense sequence of SEQ ID NO: 3 and an antisense sequence of SEQ ID NO: 4; and an siRNA having a sense sequence of SEQ ID NO: 5 and an antisense sequence of SEQ ID NO: 6.

2. The nucleic-acid pharmaceutical composition of claim 1, wherein the siRNA is a form modified chemically with phosphorothioate or boranophosphate.

3. The nucleic-acid pharmaceutical composition of claim 1, wherein the siRNA is in the form of a composite with a nucleic acid delivery system.

4. The nucleic-acid pharmaceutical composition of claim 3, wherein the nucleic acid delivery system is a cationic micelle, a cationic emulsion or a cationic liposome.

5. The nucleic-acid pharmaceutical composition of claim 4, wherein the cationic micelle, the cationic emulsion or the cationic liposome comprises at least one cationic lipid selected from the group consisting of 1,2-dioleoyl-sn-glycero-3-ethylphosphocholine (EDOPC), 1-palmitoyl-2-oleoyl-sn-glycero-3-ethylphosphocholine (EPOPC), 1,2-dimyristoyl-sn-glycero-3-ethylphosphocholine (EDMPC), 1,2-distearoyl-sn-glycero-3-ethylphosphocholine (SPC), 1,2-dipalmitoyl-sn-glycero-3-ethylphosphocholine (EDPPC), 1,2-dioleoyl-3-trimethylammonium-propane (DOTAP), N-[1-(2,3-dioleyloxy)propyl]-N,N,N-trimethylammonium chloride (DOTMA), and 3β-[N-(N′,N′-dimethylaminoethane)-carbamoyl]cholesterol (DC-Cholesterol).

6. The nucleic-acid pharmaceutical composition of claim 5, wherein the cationic micelle, the cationic emulsion or the cationic liposome further comprises at least one auxiliary lipid selected from the group consisting of 1,2-diacyl-sn-glycero-3-phosphoethanolamine (DOPE), 1,2-diphytanoyl-sn-glycero-3-phosphoethanolamine (DPhPE), 1,2-dioleoyl-sn-glycero-3-phosphocholine (DOPC), 1,2-dioleoyl-sn-glycero-3-[phospho-L-serine] (DOPS), 1,2-dioleoyl-sn-glycero-3-ethylphosphocholine (DO-Ethyl-PC), and cholesterol.

7. The nucleic-acid pharmaceutical composition of claim 3, wherein the nucleic acid delivery system is a cationic polymer.

8. The nucleic-acid pharmaceutical composition of claim 7, wherein the cationic polymer is at least one selected from the group consisting of poly-L-lysine (PLL), poly-L-ornithine, poly-L-histidine, bis(3-aminopropyl) terminated polytetrahydrofuran (AT-PTHF), polyacrylamide (PA), poly(α-[4-aminobutyl]-L-glycolic acid (PAGA), poly(2-aminoethyl propylene phosphate (PPE-EA), cationic derivatives of cyclodextrin, poly(2-(dimethylamino)ethyl methacrylate (pDMAEMA), poly(4-vinylpyridine (P4VP), O,O′-Bis(2-aminopropyl) polypropylene glycol-block-polyethylene glycol-block-polypropylene glycol, poly-N-ethyl-4-vinylpyridinium tribromide (PVP), chitosan, cationic chitosan derivatives, polyamidoamine (PAMAM), fractured PAMAM, polyethyleneimine (PEI), and polyethyleneimine derivatives.

9. The nucleic-acid pharmaceutical composition of claim 1, further comprising an anticancer chemotherapeutic agent.

10. An siRNA inhibiting expression of Mcl-1, the siRNA having a sense sequence of SEQ ID NO: 1 and an antisense sequence of SEQ ID NO: 2.

11. An siRNA inhibiting expression of Mcl-1, the siRNA having a sense sequence of SEQ ID NO: 3 and an antisense sequence of SEQ ID NO: 4.

12. An siRNA inhibiting expression of Mcl-1, the siRNA having a sense sequence of SEQ ID NO: 5 and an antisense sequence of SEQ ID NO: 6.

13. The nucleic-acid pharmaceutical composition of claim 1, further comprising at least one siRNA selected from the group consisting of siRNAs inhibiting expression of Wnt-1, Hec1, Survivin, Livin, Bcl-2, XIAP, Mdm2, EGF, EGFR, VEGF, VEGFR, GASC1, IGF1R, Akt1, Grp78, STAT3, STAT5a, β-catenin, WISP1, or c-myc.
